(19) United States
(12) Patent Application Publication
(10) Pub. No.: US 2022/0292945 A1
(43) Pub. Date: Sep. 15, 2022

(54) WHEELCHAIR SAFETY MELODY APPARATUS

(71) Applicant: Wen-Hsiung Liu, Kaohsiung City (TW)

(72) Inventor: Wen-Hsiung Liu, Kaohsiung City (TW)

(21) Appl. No.: 17/467,455

(22) Filed: Sep. 7, 2021

(30) Foreign Application Priority Data
Mar. 10, 2021 (TW) 110202541

(51) Int. Cl. G08B 21/02; A61G 5/10; G08B 3/10

(52) U.S. Cl. CPC G08B 21/023; A61G 5/1045; G08B 3/10

(57) ABSTRACT

The invention is related to a wheelchair safety melody apparatus, which comprising: a wheelchair, including a seat pad; at least one handrail, fixed to the wheelchair; a pressure-sensitive seat pad, placed above the seat pad; and a safety melody generator, electrically connected with the pressure-sensitive seat pad, and being removably fixed at one side of the at least one handrail via a fastening element, wherein as a user leaves the seat of the wheelchair, the pressure-sensitive seat pad generates an initiating signal due to release of the pressure of the pressure-sensitive seat pad, thereby to allow the safety melody generator to generate an alarm signal for notifying a medical care person for assistance.

### CROSS-REFERENCES TO RELATED APPLICATIONS

The present application claims priority to Taiwan Patent Application No. 110202541 filed on Mar. 10, 2021, which is incorporated herein by reference in its entirety.

### FIELD OF THE INVENTION

The invention is related to a wheelchair safety melody apparatus for generating an alarm melody at the time as a user under medical care by taking a seat on the wheelchair leaves or falls off from the wheelchair, thereby informing the medical care person for assistance in time.

### BACKGROUND OF THE INVENTION

Basing on the need of taking care of a wheelchair user, the equipments of wheelchair providing with an alarm function at present meet with the problems of being expensive, poor alarming efficacy, complicated structure, and poor movability due to the use of a wired power source. Therefore, it is desired to develop a wheelchair safety melody apparatus to overcome all the above problems and to be a great aid for the user to be taken care by seating on the wheelchair.

### SUMMARY OF THE INVENTION

The invention is intended to provide a wheelchair safety melody apparatus featured in cost advantage, simple structure, better performance efficacy and movability in all directions without obstruction, so as to take care of a user under medical care and seating on a wheelchair in a safe and efficient manner.

A wheelchair safety melody apparatus according to the invention comprises:

a wheelchair, including a seat pad;
at least one handrail, fixed to the wheelchair;
a pressure-sensitive seat pad, placed above the seat pad of the wheelchair; and
a safety melody generator, electrically connected with the pressure-sensitive seat pad, and being removably fixed at one side of the at least one handrail via a fastening element,
wherein as a user leaves the seat of the wheelchair, the pressure-sensitive seat pad generates an initiating signal due to release of the pressure of the pressure-sensitive seat pad, thereby to allow the safety melody generator to generate an alarm signal for notifying a medical care person.

The invention is provided with the following features.
I. The safety melody generator provides an alarming function as the user seating on the wheelchair falls off therefrom. Upon falling, the safety melody generator will be triggered to generate a musical melody due to release of the pressure from the pressure-sensitive seat pad, thereby notifying the medical care person.
   In addition, the seat pads available on the market are quite expensive. The invention develops a specific pressure-sensitive seat pad with about 50% down of the price as compared with the seat pads on the market, and thus reducing the cost tremendously.
II. The wheelchair safety melody apparatus provides a button to call for the medical care person. As the user on the wheelchair under medical care needs assistance from the medical care person, such as wishing to go to toilet or requiring certain help, he (she) may press the button to call and notify the medical care person, so as to strengthen the service efficiency of the medical care person.
III. The safety melody generator of the invention may be fixed to the at least one handrail of the wheelchair via a fastening component such as a Velcro tape so as to be fitable for use in various kinds of wheelchairs.
IV. The power consumption of the wheelchair safety melody generator is rather low and the power source may make use of the battery to facilitate moving of the wheelchair. A pair of the batteries can be utilized in about one year.

### BRIEF DESCRIPTION OF THE DRAWINGS

FIG. 1 is a schematical structure diagram of the wheelchair safety melody apparatus according to the invention.

FIG. 2 shows a schematical structure diagram of the safety melody generator of the wheelchair safety melody apparatus according to the invention.

### EMBODIMENTS OF THE INVENTION

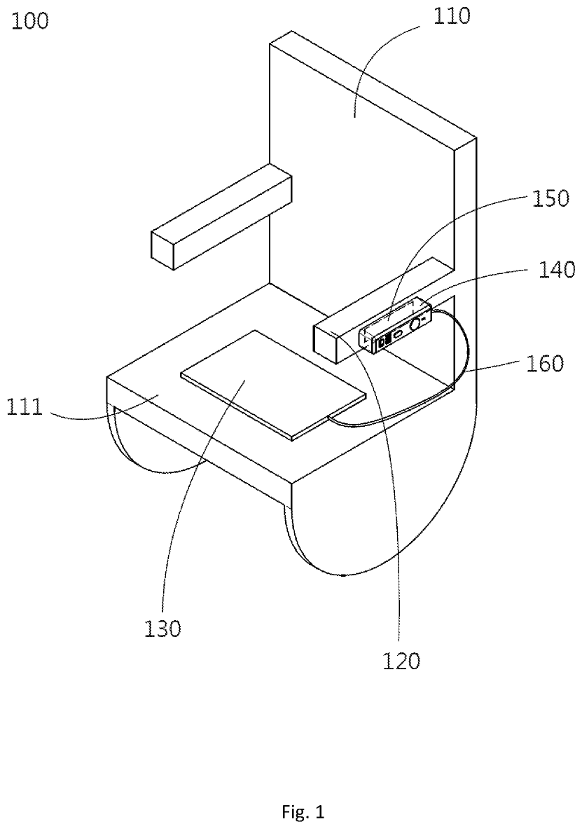

As shown in FIG. 1, a wheelchair safety melody apparatus 100 according to the invention comprises:

a wheelchair 110, including a seat pad 111;
at least one handrail 120, fixed to the wheelchair 110;
a pressure-sensitive seat pad 130, placed above the seat pad 111 of the wheelchair 110;
and
a safety melody generator 140, electrically connected with the pressure-sensitive seat pad 130, and being removably fixed at one side of the at least one handrail 120 via a fastening element 150,
wherein as a user leaves the seat of the wheelchair, the pressure-sensitive seat pad generates an initiating signal due to release of the pressure of the pressure-sensitive seat pad, thereby to allow the safety melody generator to generate an alarm signal for notifying a medical care person.

It is noted that the leaving of the user from the seat of the wheelchair is that the user is standing or sliding off from the wheelchair.

In the wheelchair safety melody apparatus 100 of the invention, the alarm signal is a musical melody or other alarming sound.

According to the wheelchair safety melody apparatus of the invention 100, the safety melody generator 140 and the pressure-sensitive seat pad 130 are electrically connected through a connection wire 160, in which a middle part of the connection wire is mutually engaged via a male connector and a female connector of a telephone wire (not shown in the drawing) to facilitate free movement of the wheelchair.

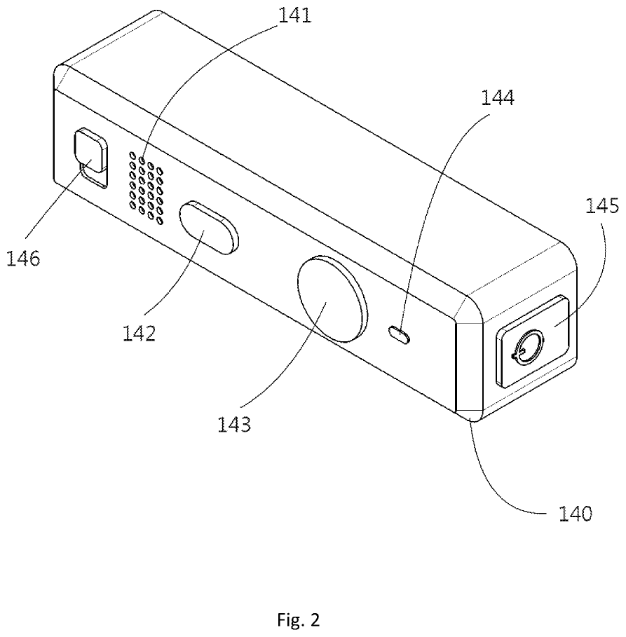

As shown in FIG. 2, in the wheelchair safety melody apparatus100 of the invention, the safety melody generator 140 includes: an alarm signal exit 141 for emitting the alarm signal; an alarm signal stop button 142 for shutting down the alarm signal; an emergency button 143 for emitting the alarm signal upon pressing so as to inform the medical care person for assistance; a power indicator 144 for generating a light indicating after power-on, and a power switch 145.

In addition, the safety melody generator 140 further includes a volume control switch 146, and a power source (not shown in the drawing) which is a battery to facilitate movement of the wheelchair.

In the wheelchair safety melody apparatus 100, the fastening element 150 is a Velcro tape or a fastening component with male and female engaging function (not shown in the drawing).

What is claimed is:

1. A wheelchair safety melody apparatus, comprising:
a wheelchair, including a seat pad;
at least one handrail, fixed to the wheelchair;
a pressure-sensitive seat pad, placed above the seat pad; and
a safety melody generator, electrically connected with the pressure-sensitive seat pad, and being removably fixed at one side of the at least one handrail via a fastening element,
wherein as a user leaves the seat of the wheelchair, the pressure-sensitive seat pad generates an initiating signal due to release of the pressure of the pressure-sensitive seat pad, thereby to allow the safety melody generator to generate an alarm signal for notifying a medical care person.

2. A wheelchair safety melody apparatus as claimed in claim 1, wherein the leaving of the user from the seat of the wheelchair is standing or sliding off from the wheelchair.

3. A wheelchair safety melody apparatus as claimed in claim 1, wherein the alarm signal is a musical melody or other alarming sound.

4. A wheelchair safety melody apparatus as claimed in claim 1, wherein the safety melody generator and the pressure-sensitive seat pad are electrically connected through a connection wire, in which a middle part of the connection wire is mutually engaged via a male connector and a female connector of a telephone wire.

5. A wheelchair safety melody apparatus as claimed in claim 3, wherein the safety melody generator includes: an alarm signal exit for emitting the alarm signal; an alarm signal stop button for shutting down the alarm signal; an emergency button for emitting the alarm signal upon pressing so as to inform the medical care person for assistance; a power indicator for generating a light indicating after power-on, and a power switch.

6. A wheelchair safety melody apparatus as claimed in claim 5, wherein the safety melody generator further includes a volume control switch.

7. A wheelchair safety melody apparatus as claimed in claim 1, wherein the fastening element is a Velcro tape or a fastening component with male and female engaging function.

8. A wheelchair safety melody apparatus as claimed in claim 1, the safety melody generator further comprises a power source which is a battery to facilitate movement of the wheelchair.
